(19) United States
(12) Patent Application Publication
(10) Pub. No.: US 2026/0034274 A1
(43) Pub. Date: Feb. 5, 2026

(54) SKIN BARRIER COMPOSITION INCLUDING TRIPLE LIPIDS

(71) Applicant: Hollister Incorporated, Libertyville, IL (US)

(72) Inventors: Jayant JOSHI, Lake Forest, IL (US); Tage CARLSON, Hawthorn Woods, IL (US)

(73) Assignee: Hollister Incorporated, Libertyville, IL (US)

(21) Appl. No.: 19/138,981

(22) PCT Filed: Feb. 17, 2025
(86) PCT No.: PCT/US2025/016238

§ 371 Date: Jun. 13, 2025

Related U.S. Application Data
(60) Provisional application No. 63/555,306, filed on Feb. 19, 2024.

(51) Int. Cl. A61L 28/00

(52) U.S. Cl. CPC A61L 28/0011; A61L 28/00; A61L 28/0019

(57) ABSTRACT

An ostomy skin barrier composition includes a water-insoluble pressure-sensitive adhesive component, a hydrophilic component, and at least three different types of lipids including ceramide, cholesterol, and fatty acids. The skin barrier composition is configured to provide skin barriers having adhesive characteristics suitable for ostomy applications to securely attach an ostomy pouch to user's peristomal skin, while minimizing peristomal skin irritation and injuries.

### BACKGROUND

The present disclosure relates to skin barrier compositions, and more particularly to skin barrier compositions for ostomy appliances including lipids.

Ostomy appliances, such as an ostomy pouch, are used to collect stoma effluent (fecal material and/or urine) for patients with a stoma created by a surgery (e.g., colostomy and ileostomy). The ostomy appliances are typically attached to user's peristomal skin via an ostomy wafer or an ostomy face plate including a skin barrier. Skin barriers are designed to securely attach and support an ostomy appliance, such as an ostomy pouch, while absorbing moisture and liquid from the peristomal skin and facilitate peristomal skin health.

Due to the continuous and prolonged use of ostomy appliances, user's peristomal skin is always at risk of irritation and injuries. Thus, it is desirable to configure skin barriers to have sufficient adhesive strength to support an ostomy pouch, yet not so aggressive as to risk skin injury or irritation during use and upon removal. Skin barriers including ceramide, such as the skin barrier available through the applicant of the present application under the trade name, CeraPlus®, have been developed to promote peristomal skin health.

The present disclosure provides improved skin barrier compositions including lipid formulations that can further reduce a risk of peristomal skin irritation and injuries and promote peristomal skin health.

### BRIEF SUMMARY

Ostomy skin barrier compositions comprising at least three different types of lipids are provided according to various embodiments. The skin barrier compositions are configured to provide skin barriers having adhesive characteristics suitable for ostomy applications to securely attach an ostomy pouch to user's peristomal skin, while minimizing peristomal skin irritation and injuries.

In one aspect, an ostomy skin barrier composition may contain a water-insoluble pressure-sensitive adhesive component, a hydrophilic component, and at least three different types of lipids including ceramide, cholesterol, and fatty acids. The at least three different types of lipids may be provided as a triple lipid formulation including ceramide, cholesterol, and fatty acid.

In an embodiment, the ostomy skin barrier composition may be configured to provide a skin barrier having a fluid absorption capacity @24 hours of about 0.4 g/cm² to about 2.0 g/cm² when tested according to ISO 12505-1, a peak tack of about 1.5 Newtons (N) to about 4.5 N when tested according to Avery Adhesive Test Method (AAT)) disclosed in Chuang et al., Avery Adhesive Test Yields More Performance Data than Traditional Probe, Adhesive Age, September 1997, and a 180° peel adhesion of about 3 N/m to about 6 N/m when tested according ISO 12505-2. For example, the ostomy skin barrier composition may be configured to provide a skin barrier having a fluid absorption capacity @24 hours of about 0.5 g/cm² to about 1.5 g/cm² when tested according to ISO 12505-1, a peak tack of about 2 N to about 4 N when tested according to Avery Adhesive Test Method (AAT), and a 180° peel adhesion of about 3.5 N/m to about 5.5 N/m when tested according to ISO 12505-2.

In an embodiment, the ostomy skin barrier composition may include about 0.15 wt. % to about 2.5 wt. % of the triple lipid formulation. For example, the ostomy skin barrier composition may include about 0.2 wt. % to about 1.8 wt. % of the triple lipid formulation. In some embodiments, the ceramide may be a synthetic pseudo-ceramide. The fatty acid may be palmitic acid, linoleic acid, stearic acid, or mixtures thereof.

The water-insoluble pressure-sensitive adhesive component may comprise polyisobutylene (PIB), hydrogenated styrene-butadiene rubber (HSBR), styrene-isoprene-styrene block copolymer (SIS), styrene-butadiene-styrene block copolymer (SBS), styrene-ethylene/propylene-styrene block copolymer (SEPS), styrene-ethylene/butylene-styrene block copolymer (SEBS), styrene-ethylene/ethylene/propylene-styrene block copolymer (SEEPS), styrene-ethylene/butylene-olefin crystal block copolymer (SEBC), mineral oil, tackifier, or mixtures thereof. The hydrophilic component may comprise gum arabic, tragacanth gum, galactan, guar gum, carob gum, karaya gum, carragheenan, pectin, agar, starch, xanthan gum, dextrin, dextran, succinoglucan, pullulan, casein, albumin, gelatin, methylcellulose, ethylcellulose, methyl hydroxy propyl cellulose, hydroxyethylcellulose, cellulose sodium sulfate, hydroxy propyl cellulose, carboxymethylcellulose, sodium carboxymethylcellulose, sodium alginate, propylene glycol alginate, polyvinyl alcohol, polyvinyl methyl ether, polyvinyl pyrrolidone, carboxy vinyl polymer, sodium polyacrylate, polyacrylamide, polyethyleneimine, sucrose, or mixtures thereof.

In an embodiment, the ostomy skin barrier composition may comprise about 40 wt. % to about 70 wt. % of water-insoluble pressure-sensitive adhesive component including HSBR, PIB, mineral oil and tackifier, about 30 wt. % to about 60 wt. % of hydrophilic component including carboxymethylcellulose (CMC) and pectin, and about 1.8 wt. % of a triple lipid formulation containing Ceramide PC104, cholesterol, and linoleic acid in a ratio of 1:3:1.

In another embodiment, the ostomy skin barrier composition may comprise about 40 wt. % to about 70 wt. % of water-insoluble pressure-sensitive adhesive component including HSBR, PIB, mineral oil and tackifier, about 30 wt. % to about 60 wt. % of hydrophilic component including CMC and pectin, and about 1.8 wt. % of a triple lipid formulation containing Ceramide PC104, cholesterol, and stearic acid in a ratio of 4:2:1.

In yet another embodiment, the ostomy skin barrier composition may comprise about 40 wt. % to about 70 wt. % of water-insoluble pressure-sensitive adhesive component including HSBR, PIB, mineral oil and tackifier, about 30 wt. % to about 60 wt. % of hydrophilic component including CMC and pectin, and about 0.6 wt. % of a triple lipid formulation containing Ceramide PC104, cholesterol, and palmitic acid in a ratio of 4:2:1.

In an embodiment, the ostomy skin barrier composition may comprise about 40 wt. % to about 70 wt. % of water-insoluble pressure-sensitive adhesive component including HSBR, PIB, mineral oil and tackifier, about 30 wt. % to about 60 wt. % of hydrophilic component including CMC and pectin, and about 1.8 wt. % of a triple lipid formulation containing Ceramide PC104, cholesterol, and palmitic acid in a ratio of 3:1:1.

In another embodiment, the ostomy skin barrier composition may comprise about 40 wt. % to about 70 wt. % of water-insoluble pressure-sensitive adhesive component including HSBR, PIB, mineral oil and tackifier, about 30 wt. % to about 60 wt. % of hydrophilic component including CMC and pectin, and about 0.6 wt. % of a triple lipid formulation containing Ceramide PC104, cholesterol, and linoleic acid in a ratio of 3:1:1.

In yet another embodiment, the ostomy skin barrier composition may comprise about 40 wt. % to about 70 wt. % of water-insoluble pressure-sensitive adhesive component including HSBR, PIB, mineral oil and tackifier, about 30 wt. % to about 60 wt. % of hydrophilic component including CMC and pectin, and about 0.6 wt. % of a triple lipid formulation containing Ceramide PC104, cholesterol, and stearic acid in a ratio of 1:3:1.

In some embodiments, the ostomy skin barrier composition may also contain glycerol. For example, the ostomy skin barrier composition may include about 2 wt. % to about 12 wt. % of glycerol.

In an embodiment, the ostomy skin barrier composition may comprise about 40 wt. % to about 70 wt. % of water-insoluble pressure-sensitive adhesive component including HSBR, PIB, mineral oil and tackifier, about 20 wt. % to about 55 wt. % of hydrophilic component including CMC and pectin, and about 5 wt. % to about 10 wt. % of glycerol, and about 1.8 wt. % of a triple lipid formulation containing Ceramide PC104, cholesterol, and palmitic acid in a ratio of 3:1:1.

In an embodiment, the ostomy skin barrier composition may comprise about 40 wt. % to about 70 wt. % of a water-insoluble pressure-sensitive adhesive component including HSBR, PIB, mineral oil and hydrogenated hydrocarbon tackifier resin, about 20 wt. % to about 55 wt. % of a hydrophilic component including CMC and pectin, and about 5 wt. % to about 10 wt. % of glycerol, and about 0.15 wt. % to about 2 wt. % of a triple lipid formulation containing Ceramide PC104, cholesterol, and palmitic acid. In such an embodiment, the ostomy skin barrier composition may contain about 0.01 wt. % to about 1.2 wt. % of Ceramide PC104, about 0.03 wt. % to about 0.5 wt. % of cholesterol, and about 0.15 wt. % to about 1.2 wt. % of palmitic acid, wherein Ceramide PC104, cholesterol, and palmitic acid are included in a ratio of 1:3:20 or 1:1:10 or 1:3:100.

In an embodiment, the ostomy skin barrier composition may comprise about 40 wt. % to about 70 wt. % of a water-insoluble pressure-sensitive adhesive component including HSBR, PIB, mineral oil and hydrogenated hydrocarbon tackifier resin, about 20 wt. % to about 55 wt. % of a hydrophilic component including CMC and pectin, and about 5 wt. % to about 10 wt. % of glycerol, about 0.01 wt. % to about 0.03 wt. % of Ceramide PC104, about 0.02 wt. % to about 0.04 wt. % of cholesterol, and about 0.1 wt. % to about 0.3 wt. % of palmitic acid.

In another embodiment, the ostomy skin barrier composition may comprise about 40 wt. % to about 70 wt. % of a water-insoluble pressure-sensitive adhesive component including HSBR, PIB, mineral oil and hydrogenated hydrocarbon tackifier resin, about 20 wt. % to about 55 wt. % of a hydrophilic component including CMC and pectin, and about 5 wt. % to about 10 wt. % of glycerol, about 0.05 wt. % to about 0.15 wt. % of Ceramide PC104, about 0.05 wt. % to about 0.15 wt. % of cholesterol, and about 0.5 wt. % to about 1.5 wt. % of palmitic acid.

In yet another embodiment, the ostomy skin barrier composition may comprise about 40 wt. % to about 70 wt. % of a water-insoluble pressure-sensitive adhesive component including HSBR, PIB, mineral oil and hydrogenated hydrocarbon tackifier resin, about 20 wt. % to about 55 wt. % of a hydrophilic component including CMC and pectin, and about 5 wt. % to about 10 wt. % of glycerol, about 0.01 wt. % to about 0.03 wt. % of Ceramide PC104, about 0.02 wt. % to about 0.04 wt. % of cholesterol, and about 0.5 wt. % to about 1.5 wt. % of palmitic acid.

Other aspects, objectives and advantages will become more apparent from the following detailed description when taken in conjunction with the accompanying drawings.

### BRIEF DESCRIPTION OF THE DRAWINGS

The benefits and advantages of the present embodiments will become more readily apparent to those of ordinary skill in the relevant art after reviewing the following detailed description and accompanying drawings, wherein:

FIG. 1 is a graph of TransEpidermal Water Loss (TEWL) measurements of a skin subject after repeated barrier applications/removals from the skin subject using skin barrier samples formed from skin barrier compositions according to various embodiments;

FIG. 2 is a graph of peak tack test results of skin barrier samples formed from skin barrier compositions according to various embodiments;

FIG. 3 is a graph of fluid absorption test results of skin barrier samples formed from skin barrier compositions according to various embodiments;

FIG. 4 is an illustration of TEWL test site locations on a user; and

FIG. 5 is a graph of TEWL measurements taken from the user test sites of FIG. 4 for skin barrier samples formed from skin barrier compositions according to various embodiments.

### DETAILED DESCRIPTION

While the present disclosure is susceptible of embodiment in various forms, there is shown in the drawings and will hereinafter be described presently preferred embodiments with the understanding that the present disclosure is to be considered an exemplification and is not intended to limit the disclosure to the specific embodiments illustrated.

In an embodiment, a skin barrier composition may comprise a water-insoluble pressure-sensitive adhesive component, a hydrophilic component, and at least three different types of lipids including ceramide, cholesterol, and fatty acids. The skin barrier composition may be configured to provide skin barriers having a fluid absorption capacity @24 hours of about 0.4 g/cm² to about 2.0 g/cm², preferably about 0.5 g/cm² to about 1.5 g/cm² when tested according to ISO 12505-1, a peak tack of about 1.5 Newtons (N) to about 4.5 N, preferably about 2 N to about 4 N when tested according to Avery Adhesive Test Method (AAT) disclosed in Chuang et al., Avery Adhesive Test Yields More Performance Data than Traditional Probe, Adhesive Age, September 1997, and a 180° peel adhesion of about 3 N/m to about 6 N/m, preferably about 3.5 N/m to about 5.5 N/m when tested according to ISO 12505-2.

Suitable polymers for the water-insoluble pressure-sensitive adhesive component may include, but are not limited to, polyisobutylene (PIB), hydrogenated styrene-butadiene rubber (HSBR), styrene-isoprene-styrene block copolymer (SIS), styrene-butadiene-styrene block copolymer (SBS), styrene-ethylene/propylene-styrene block copolymer (SEPS), styrene-ethylene/butylene-styrene block copolymer (SEBS), styrene-ethylene/ethylene/propylene-styrene block copolymer (SEEPS), and styrene-ethylene/butylene-olefin crystal block copolymer (SEBC).

Suitable materials for the hydrophilic component may include, but are not limited to, gum arabic, tragacanth gum, galactan, guar gum, carob gum, karaya gum, carragheenan, pectin, agar, starch, xanthan gum, dextrin, dextran, succinoglucan, pullulan, casein, albumin, gelatin, methylcellulose, ethylcellulose, methyl hydroxy propyl cellulose, hydroxyethylcellulose, cellulose sodium sulfate, hydroxy propyl cellulose, carboxymethylcellulose, sodium carboxymethylcellulose, sodium alginate, propylene glycol alginate, polyvinyl alcohol, polyvinyl methyl ether, polyvinyl pyrrolidone, carboxy vinyl polymer, sodium polyacrylate, polyacrylamide, and polyethyleneimine.

In an embodiment, the at least three different types of lipids may include a synthetic pseudo-ceramide, such as Ceramide PC104 (C₃₉H₇₈N₂O₅), cholesterol (C₂₇H₄₆O), and at least one fatty acid, such as palmitic acid (C₁₆H₃₂O₂), linoleic acid (C₁₈H₃₂O₂), and stearic acid (C₁₈H₃₆O₂). The at least three different types of lipids may be prepared as a triple lipid formulation including ceramide, cholesterol, and fatty acid in amount of about 0.15 weight % (wt. %) to about 2.5 wt. %, preferable, about 0.2 wt. % to about 1.8 wt. %. In some embodiments, the triple lipid formulation may include ceramide, cholesterol, and fatty acid in a ratio of about 1-5:1-5:1-100. For example, the triple lipid formulation may include ceramide, cholesterol, and fatty acid in a ratio of 3:1:1 or 1:3:1 or 4:2:1 or 1:3:20 or 1:1:10 or 1:3:100.

Skin barrier samples formed from skin barrier compositions including a triple lipid formulation according to various embodiments of the present disclosure were prepared and tested for fluid absorption capacity, pH, peak tack, 180° peel adhesion, storage modulus and erosion resistance. Each of the skin barrier compositions was formulated with the same or similar base hydrocolloid adhesive formulation comprising a water-insoluble pressure-sensitive adhesive component and a hydrophilic component, wherein the amount of the water-insoluble pressure-sensitive adhesive component was adjusted to accommodate various amounts of the triple lipid formulation.

TL1 skin barrier samples were formed from a skin barrier composition comprising 54.06 wt. % of a water-insoluble pressure-sensitive adhesive component including hydrogenated styrene butadiene rubber (HSBR), polyisobutylene (PIB), mineral oil and hydrogenated hydrocarbon tackifier resin, and 44.14 wt. % of a hydrophilic component including carboxymethylcellulose (CMC), pectin and sucrose, and 1.8 wt. % of a triple lipid formulation containing Ceramide PC104, cholesterol, and linoleic acid in a ratio of 1:3:1.

TL2 skin barrier samples were formed from a skin barrier composition comprising 54.06 wt. % of a water-insoluble pressure-sensitive adhesive component including HSBR, PIB, mineral oil and hydrogenated hydrocarbon tackifier resin, and 44.14 wt. % of a hydrophilic component including CMC, pectin and sucrose, and 1.8 wt. % of a triple lipid formulation containing Ceramide PC104, cholesterol, and stearic acid in a ratio of 4:2:1.

TL3 skin barrier samples were formed from a skin barrier composition comprising 55.26 wt. % of a water-insoluble pressure-sensitive adhesive component including HSBR, PIB, mineral oil and hydrogenated hydrocarbon tackifier resin, and 44.14 wt. % of a hydrophilic component including CMC, pectin and sucrose, and 0.6 wt. % of a triple lipid formulation containing Ceramide PC104, cholesterol, and palmitic acid in a ratio of 4:2:1.

TL4 skin barrier samples were formed from a skin barrier composition comprising 54.06 wt. % of a water-insoluble pressure-sensitive adhesive component including HSBR, PIB, mineral oil and hydrogenated hydrocarbon tackifier resin, and 44.14 wt. % of a hydrophilic component including CMC, pectin and sucrose, and 1.8 wt. % of a triple lipid formulation containing Ceramide PC104, cholesterol, and palmitic acid in a ratio of 3:1:1.

TL5 skin barrier samples were formed from a skin barrier composition comprising 55.26 wt. % of a water-insoluble pressure-sensitive adhesive component including HSBR, PIB, mineral oil and hydrogenated hydrocarbon tackifier resin, and 44.14 wt. % of a hydrophilic component including CMC, pectin and sucrose, and 0.6 wt. % of a triple lipid formulation containing Ceramide PC104, cholesterol, and linoleic acid in a ratio of 3:1:1.

TL6 skin barrier samples were formed from a skin barrier composition comprising 55.26 wt. % of a water-insoluble pressure-sensitive adhesive component including HSBR, PIB, mineral oil and hydrogenated hydrocarbon tackifier resin, and 44.14 wt. % of a hydrophilic component including CMC, pectin and sucrose, and 0.6 wt. % of a triple lipid formulation containing Ceramide PC104, cholesterol, and stearic acid in a ratio of 1:3:1.

The skin barrier samples were tested for peak tack according to the AAT method, 180° peel adhesion (“peel strength”) according to ISO 12505-2, fluid absorption capacity according to ISO 12505-1, storage modulus (G′) according to (ASTM D-4092-07), erosion resistance measured based on weight loss, and pH level according to (ISO 12505-1 Skin barrier for ostomy aids-Test methods-Part 1: Size, surface pH and water-absorbency). C+ and C− samples were also tested as control samples. C+ is a sample of CerapPlus™ skin barrier comprising 0.3 wt. % of ceramide 2, which is available from the Applicant of the present application. C− is a barrier sample free of any lipid. The test results are summarized in Table 1.

TABLE 1
Test Results for Skin Barrier Samples

| | Peak | Peel | Fluid | | | |
|---|---|---|---|---|---|---|
| | Tack | strength | absorption | G′ | Erosion | |
| Sample | (N) | (N/m) | (g/cm²) | (0.1 rad/s) | resistance | pH |
| C+ | 4.43 | 4.02 | 0.99 | 80380 | 65.3% | 4.63 |
| C− | 4.31 | 3.21 | 0.76 | 75700 | 69.7% | 4.64 |
| TL1 | 3.74 | 4.67 | 0.98 | 76220 | 67.7% | 4.62 |
| TL2 | 3.83 | 4.4 | 0.96 | 79620 | 67.6% | 4.61 |
| TL3 | 4.17 | 4.45 | 0.95 | 76460 | 66.4% | 4.62 |
| TL4 | 4.24 | 4.34 | 0.99 | 82280 | 65.5% | 4.6 |
| TL5 | 3.93 | 5.25 | 0.95 | 72020 | 67.8% | 4.62 |
| TL6 | 4.35 | 4.46 | 0.89 | 79960 | 66.9% | 4.63 |

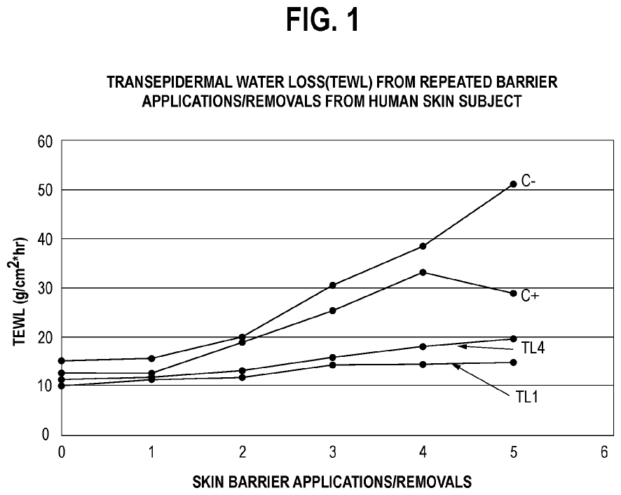

TL1 samples containing linoleic acid and TL4 samples containing palmitic acid were further tested for TransEpidermal Water Loss (TEWL) measured using TM300, a standard TEWL probe from Courage Khazaka. Impact of repeated skin barrier applications/removals on a skin was studied by repeating application and removal of TL1 barrier samples, TL4 barrier samples, C+ barrier samples, and C− barrier samples and measuring TEWL of ex vivo skin subjects after each application and removal (“stripping”) of the barrier sample. Flextend™ skin barrier, which is available from the Applicant of the present application, was used to prepare C-samples (skin barrier free of lipids). Abdominal skins derived from abdominoplasty surgeries were used as the ex vivo skin subjects. Lower TEWL values (i.e. lower water loss) can indicate less skin irritation, and thus, desirable for skin barriers. FIG. 1 is a graph of TEWL measurements from repeated skin barrier applications/removals on ex vivo skin subject #1. As shown in FIGS. 1, TL1 and TL4 samples exhibit lower TEWL when compared to C+ and C− (Flextend™) samples.

In some embodiments, the skin barrier composition may also comprise glycerol (propane-1,2,3-triol) in addition to lipids to further enhance skin hydration and fluid absorption properties of skin barriers. The skin barrier composition may comprise about 2 wt. % to about 12 wt. %, preferably about 4 wt. % to about 8 wt. % of glycerol.

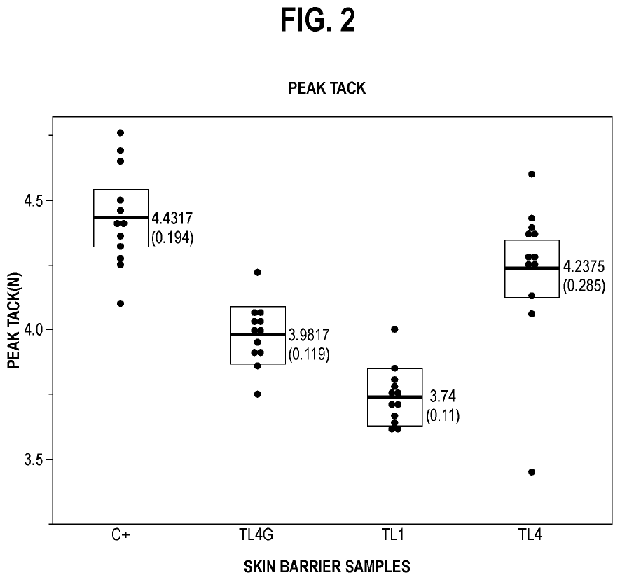

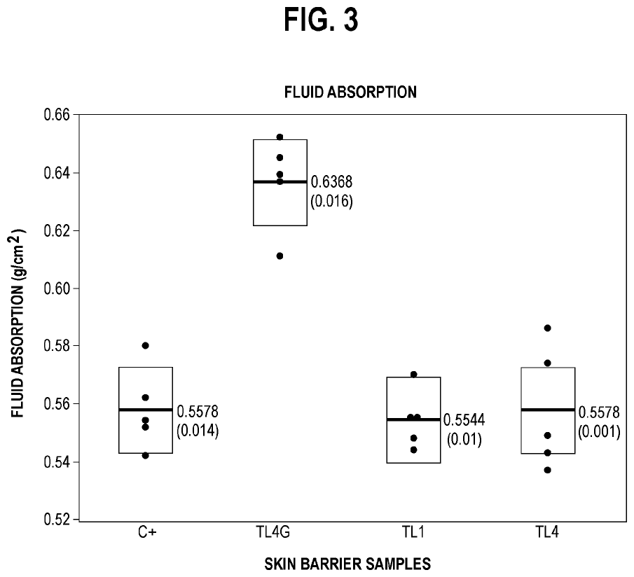

TL4G skin barrier samples were formed from a skin barrier composition comprising 54.06 wt. % of a water-insoluble pressure-sensitive adhesive component including HSBR, PIB, mineral oil and hydrogenated hydrocarbon tackifier resin, and 37.47 wt. % of a hydrophilic component including CMC and pectin, 6.67 wt. % of glycerol (also referred to herein as “glycerin”), and 1.8 wt. % of a triple lipid formulation containing Ceramide PC104, cholesterol, and palmitic acid in a ratio of 3:1:1 (1.08 wt. % Ceramide PC104, 0.36 wt. % cholesterol and 0.36 wt. % palmitic acid.) FIG. 2 is a graph of peak tack test results of TL1, TL4, TL4G, and C+ samples. FIG. 3 is a graph of fluid absorption test results of TL1, TL4, TL4G, and C+ samples. The test results and further research and development have indicated that the skin barrier compositions comprising the triple lipid formulation and glycerol may provide skin barriers having the adhesive properties suitable for ostomy skin barriers and improved fluid absorption properties without negatively impacting its erosion resistance characteristics.

User tests were conducted for TL4G skin barrier samples (“Delta barrier samples”) and TL1 skin barrier samples (“Theta barrier samples”). 32 users participated in the user tests. Each user was provided with 3 Delta barrier samples and 3 Theta barrier samples for use and evaluation. One of the users withdrew after evaluating the Theta barrier samples, thus the Delta barrier samples were evaluated by 31 users. The users were asked to evaluate and score each of the barrier samples for the following questions:
Overall acceptability—overall, how acceptable was the use of the barrier sample? Scale 1-5 (very acceptable-very unacceptable)
Application ease—how easy was it to apply the barrier sample? Scale 1-5 (very easy-very difficult)
Swelling—how much swelling of the barrier sample did you notice after removing the barrier sample? Scale 1-4 (no swelling-lots of swelling)*Rescaled from 1-4 to 1-5 for compiling average scores
Security/adherence-how securely did the barrier sample stick to your skin during wear? Scale 1-5 (very secure-very insecure)
Removal ease—how easy was it to remove the barrier sample? Scale 1-5 (very easy-very difficult).
Erosion—how much erosion of the barrier sample did you notice after removing the barrier sample after use? Scale 1-4 (no erosion-lots of erosion)*Rescaled from 1-4 to 1-5 for compiling average scores

Test results are summarized in Table 2.

TABLE 2
User Test Results for Delta and Theta Barrier Samples

| | Overall | Appli- | | | | |
|---|---|---|---|---|---|---|
| | accept- | cation | | Security/ | Removal | |
| | ability | ease | Swelling | adherence | ease | Erosion |
| Delta | 1.90 | 1.38 | 1.96 | 1.76 | 1.54 | 1.60 |
| (TL4G) | | | | | | |
| Theta | 2.14 | 1.38 | 2.19 | 1.85 | 1.64 | 1.92 |
| (TL1) | | | | | | |

The Delta barrier samples scored better than the Theta barrier samples in Overall acceptability, Swelling, Security/adherence, Removal ease and Erosion. The Delta barrier samples and the Theta barrier samples scored the same in Application ease.

Additional embodiments of the skin barrier composition comprising glycerin and palmitic acid were prepared and tested. Mix #8 skin barrier samples were formed from a skin barrier composition comprising 54.66 wt. % of a water-insoluble pressure-sensitive adhesive component including HSBR, PIB, mineral oil and hydrogenated hydrocarbon tackifier resin, and 37.47 wt. % of a hydrophilic component including CMC and pectin, 6.67 wt. % of glycerol, and 1.2 wt. % of a triple lipid formulation containing Ceramide PC104, cholesterol, and palmitic acid in a ratio of 1:1:10 (0.1 wt. % Ceramide PC104, 0.1 wt. % cholesterol and 1.0% palmitic acid). Mix #10 skin barrier samples were formed from a skin barrier composition comprising 54.82 wt. % of a water-insoluble pressure-sensitive adhesive component including HSBR, PIB, mineral oil and hydrogenated hydrocarbon tackifier resin, and 37.47 wt. % of a hydrophilic component including CMC and pectin, 6.67 wt. % of glycerol, and 1.04 wt. % of a triple lipid formulation containing Ceramide PC104, cholesterol, and palmitic acid in a ratio of 1:3:100 (0.01 wt. % Ceramide PC104, 0.03 wt. % cholesterol and 1.00% palmitic acid). Mix #17 skin barrier samples were formed from a skin barrier composition comprising 55.62 wt. % of a water-insoluble pressure-sensitive adhesive component including HSBR, PIB, mineral oil and hydrogenated hydrocarbon tackifier resin, and 37.47 wt. % of a hydrophilic component including CMC and pectin, 6.67 wt. % of glycerol, and 0.24 wt. % of a triple lipid formulation containing Ceramide PC104, cholesterol, and palmitic acid in a ratio of 1:3:20 (0.01 wt. % Ceramide PC104, 0.03 wt. % cholesterol and 0.20% palmitic acid).

Mix #8 barrier samples, Mix #10 barrier samples and Mix #17 barrier samples were tested for fluid absorption capacity according to ISO 12505-1, peak tack according to the AAT method, 180° peel adhesion (“peel strength”) according to ISO 12505-2, erosion resistance measured based on weight loss, and elastic modulus according to ASTM D4092-07 (Standard Terminology for Plastics-Dynamic Mechanical Properties). Test results are shown in Table 3.

TABLE 3
Test Results for Mix #8, 10 and 17 Barrier Samples

| | Peak | Peel | Fluid | Elastic | |
|---|---|---|---|---|---|
| | Tack | strength | absorption | modulus | Erosion |
| Sample | (N) | (N/m) | (g/cm²) | (Pa) | resistance |
| Mix #8 | 2.71 | 4.6 | 0.58 | 81560 | 65% |
| Mix #10 | 1.95 | 3.63 | 0.63 | 97420 | 64.5% |
| Mix #17 | 3.8 | 4.3 | 0.513 | 67980 | 66% |

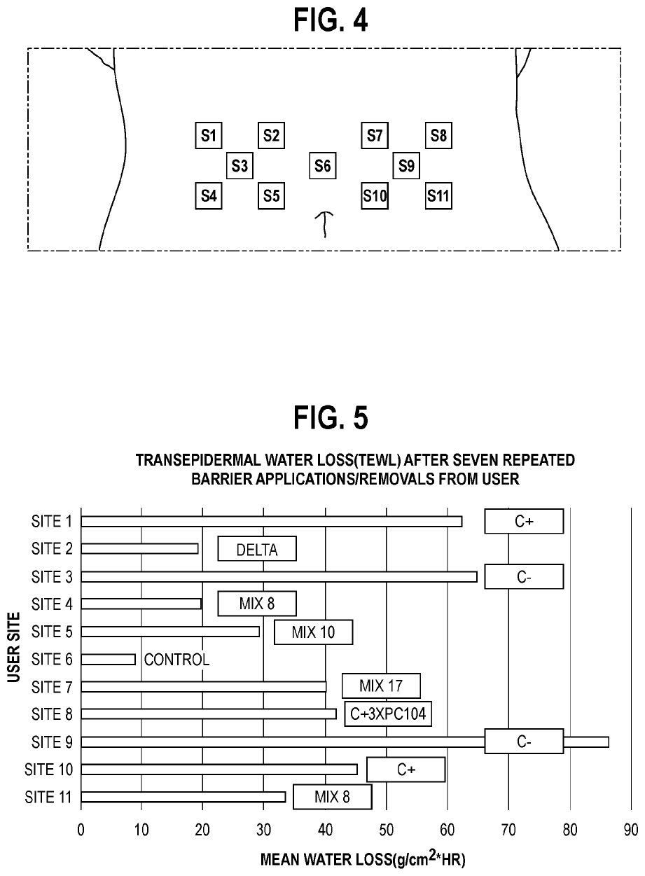

User tests were conducted to measure TEWL for Delta barrier samples, Mix #8 barrier samples, Mix #10 barrier samples, Mix #17 barrier samples, C+ barrier samples, C− barrier samples, and C+3XPC104 barrier samples. C+3XPC104 barrier samples were formed from a skin barrier composition comprising 55.21 wt. % of a water-insoluble pressure-sensitive adhesive component including HSBR, PIB, mineral oil and hydrogenated hydrocarbon tackifier resin, and 43.88 wt. % of a hydrophilic component including CMC, pectin and sucrose, and 0.91 wt. % of Ceramide PC104. FIG. 4 shows a site map of locations on a user, on which each of the barrier samples was applied and removed for TEWL measurements. TEWL of the same user without any barrier application/removal was also measured as a control.

FIG. 5 is a graph of TEWL measurements taken after repeating application and removal of the barrier samples seven times. C+ barrier samples were tested on Site 1 and Site 10 of the user. Delta barrier samples were tested on Site 2 of the user. Mix #8 barrier samples were tested on Site 4 and Site 11 of the user. Mix #10 barrier samples were tested on Site 5 on the user. Mix #17 barrier samples were tested on Site 7 of the user. C+3XPC104 barrier samples were tested on Site 8 of the user. C− barrier samples were tested on Site 3 and Site 9 of the user. Site 6 of the user was measured for TEWL without any barrier application/removal as Control. As shown in FIG. 5, Delta, Mix #8, Mix #10 and Mix #17 barrier samples, which were formed from skin barrier compositions comprising glycerin and a triple lipid formulations including palmitic acid, exhibited TEWL measurements that were lower (less water loss) than the TEWL measurements of C+, C− and C+3XPC104 barrier samples.

All patents referred to herein, are hereby incorporated herein in their entirety, by reference, whether or not specifically indicated as such within the text of this disclosure.

In the present disclosure, the words “a” or “an” are to be taken to include both the singular and the plural. Conversely, any reference to plural items shall, where appropriate, include the singular.

From the foregoing it will be observed that numerous modifications and variations can be effectuated without departing from the true spirit and scope of the novel concepts of the present disclosure. It is to be understood that no limitation with respect to the specific embodiments illustrated is intended or should be inferred. The disclosure is intended to cover by the appended claims all such modifications as fall within the scope of the claims.

What is claimed is:

1. An ostomy skin barrier composition comprising:
a water-insoluble pressure-sensitive adhesive component;
a hydrophilic component; and
at least three different types of lipids including ceramide, cholesterol, and fatty acids.

2. The ostomy skin barrier composition of claim 1, wherein the ostomy skin barrier composition is configured to provide a skin barrier having a fluid absorption capacity @24 hours of about 0.4 g/cm² to about 2.0 g/cm² when tested according to ISO 12505-1, a peak tack of about 1.5 Newtons (N) to about 4.5 N when tested according to Avery Adhesive Test Method (AAT), and a 180° peel adhesion of about 3 N/m to about 6 N/m when tested according to ISO 12505-2.

3. The ostomy skin barrier composition of claim 2, wherein the ostomy skin barrier composition is configured to provide a skin barrier having a fluid absorption capacity @24 hours of about 0.5 g/cm² to about 1.5 g/cm² when tested according to ISO 12505-1, a peak tack of about 2 N to about 4 N when tested according to Avery Adhesive Test Method (AAT), and a 180° peel adhesion of about 3.5 N/m to about 5.5 N/m when tested according to ISO 12505-2.

4. The ostomy skin barrier composition of claim 1, wherein the at least three different types of lipids are provided as a triple lipid formulation including ceramide, cholesterol, and fatty acid, wherein the ostomy skin barrier composition includes about 0.15 wt. % to about 2.5 wt. % of the triple lipid formulation.

5. The ostomy skin barrier composition of claim 1, wherein the at least three different types of lipids are provided as a triple lipid formulation including ceramide, cholesterol, and fatty acid, wherein the ostomy skin barrier composition includes about 0.2 wt. % to about 1.8 wt. % of the triple lipid formulation.

6. The ostomy skin barrier composition of claim 1, wherein the ceramide is a synthetic pseudo-ceramide.

7. The ostomy skin barrier composition of claim 1, wherein the fatty acid is palmitic acid, linoleic acid, stearic acid, or mixtures thereof.

8. The ostomy skin barrier composition of claim 1, wherein the water-insoluble pressure-sensitive adhesive component comprises polyisobutylene (PIB), hydrogenated styrene-butadiene rubber (HSBR), styrene-isoprene-styrene block copolymer (SIS), styrene-butadiene-styrene block copolymer (SBS), styrene-ethylene/propylene-styrene block copolymer (SEPS), styrene-ethylene/butylene-styrene block copolymer (SEBS), styrene-ethylene/ethylene/propylene-styrene block copolymer (SEEPS), styrene-ethylene/butylene-olefin crystal block copolymer (SEBC), mineral oil, tackifier, or mixtures thereof.

9. The ostomy skin barrier composition of claim 1, wherein the hydrophilic component comprises gum arabic, tragacanth gum, galactan, guar gum, carob gum, karaya gum, carragheenan, pectin, agar, starch, xanthan gum, dextrin, dextran, succinoglucan, pullulan, casein, albumin, gelatin, methylcellulose, ethylcellulose, methyl hydroxy propyl cellulose, hydroxyethylcellulose, cellulose sodium sulfate, hydroxy propyl cellulose, carboxymethylcellulose, sodium carboxymethylcellulose, sodium alginate, propylene glycol alginate, polyvinyl alcohol, polyvinyl methyl ether, polyvinyl pyrrolidone, carboxy vinyl polymer, sodium polyacrylate, polyacrylamide, polyethyleneimine, sucrose, or mixtures thereof.

10. The ostomy skin barrier composition of claim 1, wherein the ostomy skin barrier composition comprises about 40 wt. % to about 70 wt. % of a water-insoluble pressure-sensitive adhesive component including hydrogenated styrene-butadiene rubber (HSBR), polyisobutylene (PIB), mineral oil and hydrogenated hydrocarbon tackifier resin, about 30 wt. % to about 60 wt. % of a hydrophilic component including carboxymethylcellulose (CMC) and pectin, and about 1.8 wt. % of a triple lipid formulation containing Ceramide PC104, cholesterol, and linoleic acid in a ratio of 1:3:1.

11. The ostomy skin barrier composition of claim 1, wherein the ostomy skin barrier composition comprises about 40 wt. % to about 70 wt. % of a water-insoluble pressure-sensitive adhesive component including HSBR, PIB, mineral oil and hydrogenated hydrocarbon tackifier resin, about 30 wt. % to about 60 wt. % of a hydrophilic component including CMC and pectin, and about 1.8 wt. % of a triple lipid formulation containing Ceramide PC104, cholesterol, and stearic acid in a ratio of 4:2:1.

12. The ostomy skin barrier composition of claim 1, wherein the ostomy skin barrier composition comprises about 40 wt. % to about 70 wt. % of a water-insoluble pressure-sensitive adhesive component including HSBR, PIB, mineral oil and hydrogenated hydrocarbon tackifier resin, about 30 wt. % to about 60 wt. % of a hydrophilic component including CMC and pectin, and about 0.6 wt. % of a triple lipid formulation containing Ceramide PC104, cholesterol, and palmitic acid in a ratio of 4:2:1.

13. The ostomy skin barrier composition of claim 1, wherein the ostomy skin barrier composition comprises about 40 wt. % to about 70 wt. % of a water-insoluble pressure-sensitive adhesive component including HSBR, PIB, mineral oil and hydrogenated hydrocarbon tackifier resin, about 30 wt. % to about 60 wt. % of a hydrophilic component including CMC and pectin, and about 1.8 wt. % of a triple lipid formulation containing Ceramide PC104, cholesterol, and palmitic acid in a ratio of 3:1:1.

14. The ostomy skin barrier composition of claim 1, wherein the ostomy skin barrier composition comprises about 40 wt. % to about 70 wt. % of a water-insoluble pressure-sensitive adhesive component including HSBR, PIB, mineral oil and hydrogenated hydrocarbon tackifier resin, about 30 wt. % to about 60 wt. % of a hydrophilic component including CMC and pectin, and about 0.6 wt. % of a triple lipid formulation containing Ceramide PC104, cholesterol, and linoleic acid in a ratio of 3:1:1.

15. The ostomy skin barrier composition of claim 1, wherein the ostomy skin barrier composition comprises about 40 wt. % to about 70 wt. % of a water-insoluble pressure-sensitive adhesive component including HSBR, PIB, mineral oil and hydrogenated hydrocarbon tackifier resin, about 30 wt. % to about 60 wt. % of a hydrophilic component including CMC and pectin, and about 0.6 wt. % of a triple lipid formulation containing Ceramide PC104, cholesterol, and stearic acid in a ratio of 1:3:1.

16. The ostomy skin barrier composition of claim 1, further comprising glycerol.

17. The ostomy skin barrier composition of claim 16, wherein the ostomy skin barrier composition comprises about 2 wt. % to about 12 wt. % of glycerol.

18. The ostomy skin barrier composition of claim 17, wherein the ostomy skin barrier composition comprises about 40 wt. % to about 70 wt. % of a water-insoluble pressure-sensitive adhesive component including HSBR, PIB, mineral oil and hydrogenated hydrocarbon tackifier resin, about 20 wt. % to about 55 wt. % of a hydrophilic component including CMC and pectin, and about 5 wt. % to about 10 wt. % of glycerol, and about 1.8 wt. % of a triple lipid formulation containing Ceramide PC104, cholesterol, and palmitic acid in a ratio of 3:1:1.

19. The ostomy skin barrier composition of claim 1, wherein the ostomy skin barrier composition comprises about 40 wt. % to about 70 wt. % of a water-insoluble pressure-sensitive adhesive component including HSBR, PIB, mineral oil and hydrogenated hydrocarbon tackifier resin, about 20 wt. % to about 55 wt. % of a hydrophilic component including CMC and pectin, and about 5 wt. % to about 10 wt. % of glycerol, and about 0.15 wt. % to about 2 wt. % of a triple lipid formulation containing Ceramide PC104, cholesterol, and palmitic acid.

20. The ostomy barrier composition of claim 19, wherein the ostomy skin barrier composition contains about 0.01 wt. % to about 1.2 wt. % of Ceramide PC104, about 0.03 wt. % to about 0.5 wt. % of cholesterol, and about 0.15 wt. % to about 1.2 wt. % of palmitic acid.

21. The ostomy barrier composition of claim 19, wherein the triple lipid formulation includes Ceramide PC104, cholesterol, and palmitic acid in a ratio of 1:3:20.

22. The ostomy barrier composition of claim 19, wherein the triple lipid formulation includes Ceramide PC104, cholesterol, and palmitic acid in a ratio of 1:1:10.

23. The ostomy barrier composition of claim 19, wherein the triple lipid formulation includes Ceramide PC104, cholesterol, and palmitic acid in a ratio of 1:3:100.

24. The ostomy skin barrier composition of claim 1, wherein the ostomy skin barrier composition comprises about 40 wt. % to about 70 wt. % of a water-insoluble pressure-sensitive adhesive component including HSBR, PIB, mineral oil and hydrogenated hydrocarbon tackifier resin, about 20 wt. % to about 55 wt. % of a hydrophilic component including CMC and pectin, and about 5 wt. % to about 10 wt. % of glycerol, about 0.01 wt. % to about 0.03 wt. % of Ceramide PC104, about 0.02 wt. % to about 0.04 wt. % of cholesterol, and about 0.1 wt. % to about 0.3 wt. % of palmitic acid.

25. The ostomy skin barrier composition of claim 1, wherein the ostomy skin barrier composition comprises about 40 wt. % to about 70 wt. % of a water-insoluble pressure-sensitive adhesive component including HSBR, PIB, mineral oil and hydrogenated hydrocarbon tackifier resin, about 20 wt. % to about 55 wt. % of a hydrophilic component including CMC and pectin, and about 5 wt. % to about 10 wt. % of glycerol, about 0.05 wt. % to about 0.15 wt. % of Ceramide PC104, about 0.05 wt. % to about 0.15 wt. % of cholesterol, and about 0.5 wt. % to about 1.5 wt. % of palmitic acid.

26. The ostomy skin barrier composition of claim 1, wherein the ostomy skin barrier composition comprises about 40 wt. % to about 70 wt. % of a water-insoluble pressure-sensitive adhesive component including HSBR, PIB, mineral oil and hydrogenated hydrocarbon tackifier resin, about 20 wt. % to about 55 wt. % of a hydrophilic component including CMC and pectin, and about 5 wt. % to about 10 wt. % of glycerol, about 0.01 wt. % to about 0.03 wt. % of Ceramide PC104, about 0.02 wt. % to about 0.04 wt. % of cholesterol, and about 0.5 wt. % to about 1.5 wt. % of palmitic acid.
